(19) United States
(12) Patent Application Publication
(10) Pub. No.: US 2025/0073144 A1
(43) Pub. Date: Mar. 6, 2025

(54) COSMETIC COMPOSITION FOR ENHANCING SKIN ABSORPTION

(71) Applicant: COSMAX, INC., Gyeonggi-Do (KR)

(72) Inventors: Ji Hyun LEE, Gyeonggi-do (KR); Min Joo NOH, Gyeonggi-Do (KR); Jun Bae LEE, Gyeonggi-Do (KR); Myeong Sam PARK, Gyeonggi-Do (KR)

(73) Assignee: COSMAX, INC., Gyeonggi-Do (KR)

(21) Appl. No.: 18/293,249

(22) PCT Filed: Sep. 29, 2022
(86) PCT No.: PCT/KR2022/014654

§ 371 Date: Jan. 29, 2024

(30) Foreign Application Priority Data
Sep. 30, 2021 (KR) 10-2021-0130299

(51) Int. Cl. A61K 8/36; A61K 8/42; A61K 8/55; A61K 8/60; A61K 8/63; A61K 8/67; A61Q 19/02; A61Q 19/08

(52) U.S. Cl. CPC A61K 8/361; A61K 8/42; A61K 8/553; A61K 8/606; A61K 8/63; A61K 8/675; A61Q 19/02; A61Q 19/08; A61K 2800/412

(57) ABSTRACT

This relates to a cosmetic composition, which includes a skin carrier that has a skin-like structure, and may improve or enhance the absorption of an active ingredient into the skin.

### TECHNICAL FIELD

The present application claims priority to Korean Patent Application No. 10-2021-0130299, filed on Sep. 30, 2021, which is hereby incorporated by reference in its entirety.

The present application relates to a cosmetic composition.

### BACKGROUND ART

The stratum corneum of the skin performs a skin barrier function to prevent external harmful substances and transepidermal moisture loss, and has been identified as a key pathway for skin absorption. There are three known pathways for skin absorption of effective ingredients, an intracellular pathway, an intercellular pathway, and a follicular pathway, of which the intercellular pathway predominantly occurs. Intercellular lipids, a main absorption pathway, are composed of ceramides, cholesterol, free fatty acids, cholesterol sulfate, and phospholipids, and a cosmetic with the intercellular lipids and similar ingredients may act as a skin lipid enhancer to help promote skin absorption.

In order to improve the skin absorption promotion of a bioactive substance, research has been conducted on various formulations with biocompatibility and skin-like structures, but most of them are limited to liposomes or an emulsified formulation. Meanwhile, solubilized formulations may be applied to a variety of formulations across cosmetics, but the effect of promoting skin absorption may be reduced due to a small oil phase portion content.

Therefore, in order to increase the skin absorption rate of an active ingredient, it is necessary to develop a skin carrier of a solubilized formulation that has excellent biocompatibility and a skin-like structure and may promote skin absorption through interaction with stratum corneum intercellular lipids.

### DISCLOSURE

### Technical Problem

One aspect is to provide a cosmetic composition comprising a nanoparticle carrier comprising ceramide; hydrogenated lecithin; fatty acids including stearic acid and oleic acid; and a cholesterol derivative.

Another aspect is to provide a use of a composition comprising the nanoparticle carrier comprising the ceramide; the hydrogenated lecithin; the fatty acid including stearic acid and oleic acid; and the cholesterol derivative, for making a cosmetic product.

### Technical Solution

One aspect provides a cosmetic composition including a nanoparticle carrier including a ceramide; a hydrogenated lecithin; a fatty acid containing stearic acid and oleic acid; and a cholesterol derivative.

As used herein, the term “nanoparticle carrier” refers to a ceramide-based skin carrier having a structure similar to a skin lipid, which has the effect of promoting skin absorption of an active ingredient. Therefore, the cosmetic composition according to an aspect may be for promoting or improving the skin absorption of an active substance. In addition, the cosmetic composition may form a lamellar structure similar to the structure of the stratum corneum of the skin, thereby improving skin affinity and moisturizing power, and may improve skin barrier function.

The nanoparticle carrier may have an average particle size of 100 nm to 300 nm. The nanoparticle carrier may have a size of, for example, 100 nm to 300 nm, 150 nm to 200 nm, 155 nm to 185 nm, 155 nm to 183 nm, 160 nm to 180 nm, 165 nm to 185 nm, 170 nm to 175 nm, or 180 nm to 185 nm. In this case, when the average particle size of the nanoparticle carrier is less than the above range, the nanoparticle carrier may cause skin toxicity, and when the average particle size is greater than the above range, the stability of the nanoparticle carrier is reduced, interparticle aggregation occurs, and an emulsified particle is formed to form an opaque formulation. The nanoparticle carrier may be appropriately adjusted according to the content of lecithin, ceramide and fatty acid, and by having a size in the above range, a translucent formulation may be formed, as well as being conducive to stability over time. In addition, the translucent formulations may exhibit a structure similar to a skin lipid, thereby promoting the absorption of the active ingredient into the skin.

The ceramide may be included at 0.01 wt % to 0.1 wt %, based on the total weight of the composition. The ceramide may include, for example, 0.01 wt % to 0.1 wt %, 0.02 wt % to 0.09 wt %, 0.03 wt % to 0.08 wt %, 0.03 wt % to 0.07 wt %, or 0.04 wt % to 0.06 wt %, based on the total weight of the composition. The problem is that when a content of ceramide is less than the above range, the temporal stability of the formulation may be affected, and when the content of ceramide is greater than the above range, the ceramide may precipitate.

The hydrogenated lecithin may include from 0.1 wt % to 0.4 wt %, based on the total weight of the composition. The hydrogenated lecithin may include, for example, 0.1 wt % to 0.4 wt %, 0.1 wt % to 0.35 wt %, 0.1 wt % to 0.3 wt %, 0.2 wt % to 0.4 wt %, or 0.25 wt % to 0.35 wt %, based on the total weight of the composition. In this case, when the content of hydrogenated lecithin is less than the above range, there is a problem that the temporal stability of the lipid particles may be affected, as well as a phenomenon of agglomeration of the lipid nanoparticles may occur, and when the content of hydrogenated lecithin is greater than the above range, there is a problem that the size of the lipid nanoparticle may increase and the transparency of the formulation may decrease.

The composition may include 0.01 wt % to 0.07 wt % oleic acid, based on the total weight of the composition. The oleic acid may include, for example, 0.01 wt % to 0.07 wt %, 0.01 wt % to 0.06 wt %, 0.01 wt % to 0.04 wt %, 0.02 wt % to 0.06 wt %, or 0.02 wt % to 0.05 wt %, based on the total weight of the composition. In this case, when the content of the oleic acid is less than the above range, there is a problem that the transparency is excessively increased due to the small size of the lipid nanoparticles, and when the content of the oleic acid is greater than the above range, there is a problem that the transparency is excessively decreased due to the precipitation of oleic acid. In other words, the composition according to an aspect may form a translucent formulation with temporal stability by including oleic acid in a certain content.

The fatty acid, including stearic acid and oleic acid, may include from 0.01 wt % to 0.2 wt % based on the total weight of the composition. The fatty acid may include, for example, 0.01 wt % to 0.2 wt %, 0.01 wt % to 0.15 wt %, 0.01 wt % to 0.1 wt %, 0.01 wt % to 0.09 wt %, 0.02 wt % to 0.18 wt %, 0.02 wt % to 0.1 wt %, 0.02 wt % to 0.08 wt %, 0.03 wt % to 0.07 wt %, or 0.04 wt % to 0.06 wt %, based on the total weight of the composition. In this case, when the content of the fatty acid is less than the above range, there is a problem that the ceramide is difficult to stabilize, and the stability of the formulation is affected by the change of curing time, and when the content of the fatty acid is greater than the above range, there is a problem that the fatty acids are precipitated and affect the opacity. In an embodiment, the stearic acid and oleic acid may be mixed in a weight ratio of 1:0.01 to 99.

In an embodiment, a cosmetic composition including a ceramide; hydrogenated lecithin; a fatty acid containing stearic acid and oleic acid; and a nanoparticle carrier including a cholesterol derivative was found to have a superior effect on improving skin wrinkles compared to a composition without hydrogenated lecithin and fatty acid. Therefore, the cosmetic composition according to an aspect may have a function of improving skin wrinkles and enhancing skin moisturization.

In an embodiment, the cosmetic composition may additionally include a skin active substance. The skin active substance may be, for example, niacinamide, adenosine, Centella asiatica, oleanolic acid, retinol, ascorbic acid, flavonoid, polyphenol, isoflavone, etc. In an embodiment, a cosmetic composition including a ceramide; hydrogenated lecithin; a fatty acid containing stearic acid and oleic acid; and a nanoparticle carrier including a cholesterol derivative and niacinamide as an active substance was found to have a superior effect on improving skin whitening to a composition without hydrogenated lecithin and fatty acid. Therefore, the cosmetic composition according to an aspect may have the effect of promoting or improving skin absorption of the skin active substance, thereby improving the functionality of the active substance.

### Advantageous Effects

The compositions according to an aspect may have a skin-like structure, thereby improving or enhancing the absorption of the active ingredient into the skin.

### DESCRIPTION OF DRAWINGS

FIG. 1 is an observational photograph of a particle structure and appearance of a cosmetic composition according to an embodiment.

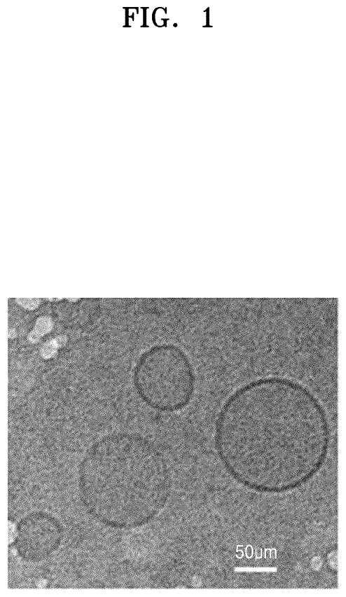

FIG. 2A is an observational photograph showing the transparency of cosmetic compositions according to fatty acid content, according to an embodiment.

FIG. 2B is an observational photograph showing the transparency according to type of fatty acids of a cosmetic composition according to an embodiment.

FIG. 3A is a small angle X-ray scattering graph of a cosmetic composition according to an embodiment.

FIG. 3B is a wide angle X-ray scattering graph of a cosmetic composition according to an embodiment.

FIG. 4A is a graph confirming the effect of improving skin permeation through an artificial membrane (strat-M) of a cosmetic composition according to an embodiment.

FIG. 4B is a graph confirming the effect of improving skin permeation through a human skin model (Epi-TEM) of a cosmetic composition according to an embodiment.

FIG. 5 is photographs confirming a wrinkle improvement effect of a cosmetic composition according to an embodiment.

FIG. 6 is graphs confirming the change rate of the overall size of wrinkles, the average depth of wrinkles, and the maximum depth of wrinkles before and after using a cosmetic composition according to an embodiment.

FIG. 7 is photographs confirming the effect of enhancing skin efficacy of a cosmetic composition according to an embodiment.

FIG. 8 is photographs confirming the effect of reducing melanin pigment in a cosmetic composition according to an embodiment.

FIG. 9 is a graph confirming the change rate of a melanin index and pigmentation area (pixel) before and after using a cosmetic composition according to an embodiment.

### BEST MODE

Hereinafter, desirable embodiments will be presented to facilitate the understanding of the present disclosure. However, the following embodiments are provided only to make the present disclosure easier to understand, and the contents of the present disclosure are not limited by the following embodiments.

### EXAMPLE

### Example 1 to 3. Preparation of Cosmetic Composition Including Lipid-Based Carrier Particle

A cosmetic composition including a lipid-based carrier particle including an ingredient and content of Table 1 below was prepared. Specifically, the ingredients of Phase B (oil phase portion) were weighed in a beaker according to their respective contents and dissolved by heating at 70° C. to 80° C. The ingredients of Phase A (aqueous phase portion) were weighed in a beaker according to their respective contents and dissolved by heating at 70° C. to 80° C. Then, phase B was gradually added to phase A and stirred continuously at 140 rpm for 10 minutes, and then cooled to 30° C. to remove air bubbles. As used herein, unless otherwise noted, the contents are in wt %.

TABLE 1

| | Content (wt %) | | | | | |
|---|---|---|---|---|---|---|
| | Ex- | Ex- | Ex- | Ex- | Ex- | Ex- |
| | ample | ample | ample | ample | ample | ample |
| Ingredient | 1 | 2 | 3 | 4 | 5 | 6 |
| Purified | To 100 | To 100 | To 100 | To 100 | To 100 | To 100 |
| water | | | | | | |
| Butylene | 5 | 5 | 5 | 5 | 5 | 5 |
| glycol | | | | | | |
| Niacinamide | — | — | — | — | — | 2 |
| Adenosine | — | — | — | — | — | 0.04 |
| Preservative | 1.3 | 1.3 | 1.3 | 1.3 | 1.3 | 1.3 |
| Ceramide | 0.05 | 0.05 | 0.05 | 0.05 | 0.05 | 0.05 |
| Hydrogenated | 0.2 | 0.2 | 0.2 | 0.2 | 0.2 | 0.2 |
| lecithin | | | | | | |
| Cholesterol | 0.1 | 0.1 | 0.1 | 0.1 | 0.1 | 0.1 |
| derivative | | | | | | |
| Dipropylene | 5 | 5 | 5 | 5 | 5 | 5 |
| glycol | | | | | | |
| Stearic acid | 0.03 | — | — | — | 0.03 | 0.03 |
| (C18:0) | | | | | | |
| Oleic acid | — | 0.02 | 0.04 | 0.06 | 0.03 | 0.03 |
| (C18:1) | | | | | | |

### COMPARATIVE EXAMPLE

### Comparative Example 1 to 10. Preparation of Cosmetic Composition Including Lipid-Based Carrier Particles

The cosmetic compositions were prepared with the same method as the above examples, except that the ingredients and contents of Table 2 below were used.

TABLE 2

| | | Content (wt %) | | | | |
|---|---|---|---|---|---|---|
| | | Comparative | Comparative | Comparative | Comparative | Comparative |
| | Ingredient | Example 1 | Example 2 | Example 3 | Example 4 | Example 5 |
| A | Purified water | To 100 | To 100 | To 100 | To 100 | To 100 |
| | Butylene glycol | 5 | 5 | 5 | 5 | 5 |
| | Niacinamide | — | — | — | — | — |
| | Adenosine | — | — | — | — | — |
| | Preservative | 1.3 | 1.3 | 1.3 | 1.3 | 1.3 |
| B | Ceramide | 0.05 | | 0.05 | 0.05 | 0.05 |
| | Hydrogenated | — | 0.2 | 0.2 | 0.2 | 0.2 |
| | lecithin | | | | | |
| | Cholesterol | 0.1 | 0.1 | 0.1 | 0.1 | 0.1 |
| | derivative | | | | | |
| | Dipropylene glycol | 5 | 5 | 5 | 5 | 5 |
| | Myristic acid | — | — | — | — | — |
| | (C14:0) | | | | | |
| | Palmitic acid | — | — | — | — | — |
| | (C16:0) | | | | | |
| | Stearic acid | — | — | — | — | — |
| | (C18:0) | | | | | |
| | Oleic acid | — | — | — | 0.08 | 0.1 |
| | (C18:1) | | | | | |
| | | Content (wt %) | | | | |
| | | Comparative | Comparative | Comparative | Comparative | Comparative |
| | Ingredient | Example 6 | Example 7 | Example 8 | Example 9 | Example 10 |
| A | Purified water | To 100 | To 100 | To 100 | To 100 | To 100 |
| | Butylene glycol | 5 | 5 | 5 | 5 | 5 |
| | Niacinamide | — | — | — | — | 2 |
| | Adenosine | — | — | — | — | 0.04 |
| | Preservative | 1.3 | 1.3 | 1.3 | 1.3 | 1.3 |
| B | Ceramide | 0.05 | 0.05 | 0.05 | 0.05 | — |
| | Hydrogenated | — | 0.2 | 0.2 | 0.2 | 0.2 |
| | lecithin | | | | | |
| | Cholesterol | 0.1 | 0.1 | 0.1 | 0.1 | 0.1 |
| | derivative | | | | | |
| | Dipropylene glycol | 5 | 5 | 5 | 5 | 5 |
| | Myristic acid | 0.06 | — | — | — | |
| | (C14:0) | | | | | |
| | Palmitic acid | — | 0.06 | — | — | |
| | (C16:0) | | | | | |
| | Stearic acid | — | — | 0.06 | — | — |
| | (C18:0) | | | | | |
| | Oleic acid | — | — | — | 0.06 | — |
| | (C18:1) | | | | | |

### EXPERIMENTAL EXAMPLE

### Experimental Example 1. Evaluation of Physical Property of Cosmetic Composition Including Lipid-Based Carrier Particle

### 1-1. Particle Size and Zeta-Potential in the Presence and Absence of Ceramide, Lecithin, and Fatty Acid.

To determine the effect of the ceramide, lecithin and fatty acid on the stability of the formulations, the particle size distribution and zeta-potential of Example 1, Example 4 and Comparative Example 1 to Comparative Example 3, and Comparative Example 5 were measured for 4 weeks. Particle analysis and zeta-potential were measured using a dynamic light scattering apparatus (DLS, SZ-100, HORIBA) under neutral (pH 7) conditions, and the results were shown in Table 3 below.

TABLE 3

| | | Example | Example | Comparative | Comparative | Comparative | Comparative |
|---|---|---|---|---|---|---|---|
| | | 1 | 4 | Example 1 | Example 2 | Example 3 | Example 5 |
| Zeta- | Week 0 | −54.6 | −62.2 | −26.1 | −49.1 | −43.5 | −63.1 |
| potential | Week 4 | −52.5 | −55.9 | −15.9 | −40.5 | −46.7 | −51.5 |
| (mV) | | | | | | | |
| Particle | Week 0 | 125.4 | 116.1 | 63.6 | 71.8 | 102.3 | 150.0 |
| size | Week 4 | 124.9 | 124.5 | 309.1 | 65.5 | 88.5 | 143.1 |
| (nm) | Particle | −0.4 | 6.75 | 79.4 | −9.61 | −15.6 | −4.82 |
| | size | | | | | | |
| | change | | | | | | |
| | rate (%) | | | | | | |

As a result, as shown in Table 3, it may be determined that Example 1, Example 4, and Comparative Example 5, which include all of ceramide, lecithin, and fatty acid, tend to exhibit a smaller particle size change rate of an emulsion formulation and a relatively higher absolute value of zeta-potential compared to Comparative Example 1 to Comparative Example 3, which do not include lecithin, fatty acid, and/or ceramide. Specifically, for Example 1, it may be determined that the particle size change rate was −0.4%, which was significantly lower than that of Example 4 or Comparative Example 5 with higher fatty acid content. In particular, in the case of Comparative Example 5, which has the highest fatty acid content, there is a possibility that a haze phenomenon may appear or lipid particles may form aggregates.

In other words, by containing ceramide, lecithin, and fatty acid, the cosmetic composition according to an aspect may form a stable formulation. In addition, by including fatty acid in a certain content, the interaction between the fatty acid and lipid composition may be controlled so that agglomeration, creaming, coalescence, etc. do not occur, and a stable formulation may be formed.

### 1-2. Particle Size and Zeta-Potential According to Type of Fatty Acid

In order to confirm the stability of the formulation according to the length of the fatty acid chain, the particle size distribution and zeta-potential of Example 5 and Comparative Example 6 to Comparative Example 9 were measured by the same method as Experimental Example 1-1 above, and the results were shown in Table 4 below.

TABLE 4

| | | Example | Comparative | Comparative | Comparative | Comparative |
|---|---|---|---|---|---|---|
| | | 5 | Example 6 | Example 7 | Example 8 | Example 9 |
| Zeta- | Week 0 | −48.4 | −39.1 | −44.1 | −45.1 | −43.4 |
| potential | Week 4 | −45.9 | −32.5 | −34.7 | −34.9 | −36.5 |
| (mV) | | | | | | |
| Particle size | Week 0 | 182.4 | 186.2 | 194.6 | 196.1 | 151.6 |
| (nm) | Week 4 | 184.2 | 204.1 | 282.9 | 247.1 | 155.8 |
| | Particle | 0.9 | 8.69 | 30.58 | 20.65 | 2.7 |
| | size | | | | | |
| | change | | | | | |
| | rate (%) | | | | | |

As a result, as shown in Table 4, in the case of Comparative Example 6 to Comparative Example 8, which include saturated fatty acids such as myristic acid, palmitic acid, and stearic acid, the particle size tended to increase according to the increasing length of the fatty acid chain. On the other hand, in the case of Comparative Example 9 including unsaturated fatty acids such as oleic acid, it was confirmed that the size decreased even as the alkyl group increased. For Example 5, which includes a saturated fatty acid (stearic acid) and an unsaturated fatty acid (oleic acid), it was confirmed that the particle size was smaller than the particle size of Comparative Example 8, which includes stearic acid, but larger than that of Comparative Example 9, which includes oleic acid. These results suggest that oleic acid penetrates the emulsion interface due to a double bond of the oleic acid, which affects the size, softening, and permeability of the emulsion.

In other words, the cosmetic composition according to an aspect may include both saturated fatty acid and unsaturated fatty acid to form a stable formulation.

### 1-3. Confirmation of Particle Size and Transparency According to Content and Type of Fatty Acid

Particle size and transparency according to the content and type of fatty acid were confirmed. Specifically, a certain amount of Example 1 to Example 2 and Comparative Example 4 to Comparative Example 5 and Comparative Example 8 was placed in a container, and an exterior photograph was taken against a black background and a white paper with printed letters. Then, the transparency was confirmed by visually observing the extent to which the colors on the black background and the letters on the white background were reflected in the photograph.

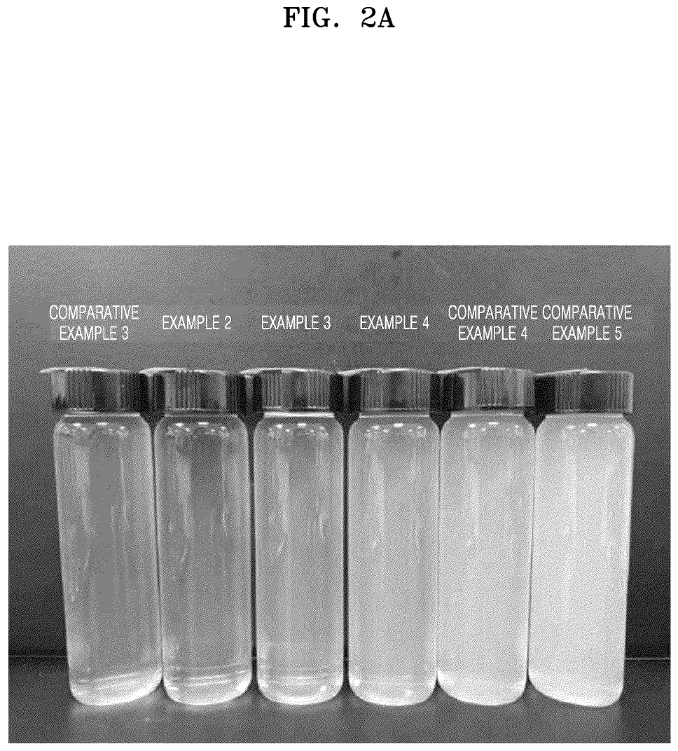

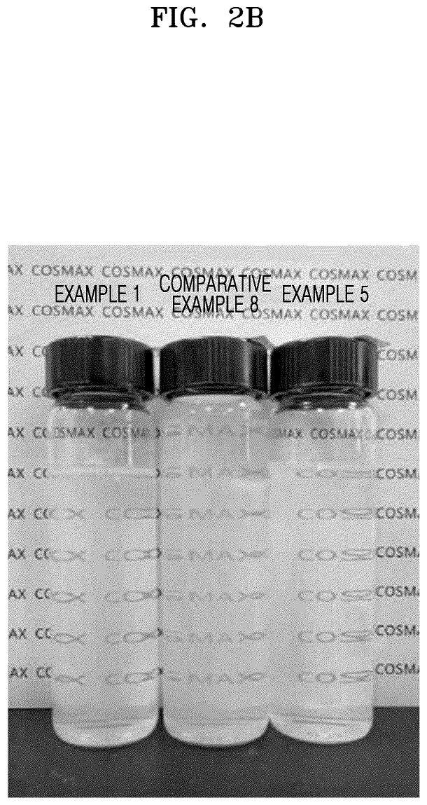

FIG. 2A is an observational photograph of the transparency of a cosmetic composition according to a fatty acid content according to an embodiment.

FIG. 2B is an observational photograph of the transparency according to a type of fatty acid of a cosmetic composition according to an embodiment.

As a result, as shown in FIG. 2A, Comparative Example 3, which does not include an oleic acid, exhibits a transparent appearance, while Comparative Example 4 to Comparative Example 5, which contain a large amount of oleic acid, exhibit an opaque appearance. On the other hand, Example 2 to Example 4, which included oleic acid at a specific content, exhibited a translucent appearance. In addition, as shown in FIG. 2B, for Example 5 and Comparative Example 8, which contained the same content of fatty acid, it was confirmed that a decrease in transparency was observed in Comparative Example 8, which did not include the oleic acid, as the letters written on the paper appeared to be crushed. In other words, it is essential to include oleic acid in order to form a translucent formulation.

Therefore, by including oleic acid in a specific amount in the cosmetic composition according to an aspect, the size of the particles is increased to a certain size, and the transparency is improved to form a translucent formulation.

### 1-3. Confirmation of Particle Structure and Appearance

Cryo-TEM (ryogenic transmission electron microscopy) was used to observe the appearance of the formulation. Specifically, 5 μl of the composition of Example 5 was loaded onto a 200 mesh carbon lacey film Cu-grid and then rapidly frozen by immersion in liquefied (about −170° C.) ethane using a vitrobot. The frozen sample was observed by Cryo-TEM (Tecnai F20, FEI) under an acceleration voltage of 200 kV.

FIG. 2 is an observational photograph of a particle structure and appearance of a cosmetic composition according to an embodiment.

As a result, as shown in FIG. 2, it was confirmed that Example 5 exhibited spherical particles and formed a bi-layer structure.

### Experimental Example 2. Crystal Structure Analysis

To confirm the emulsion structure of the cosmetic composition according to an aspect, small angle X-ray scattering (SAXS) analysis and wide angle X-ray scattering (WAXS) analysis (BL4C SAXS, Pohang Accelerator Research Center) were performed.

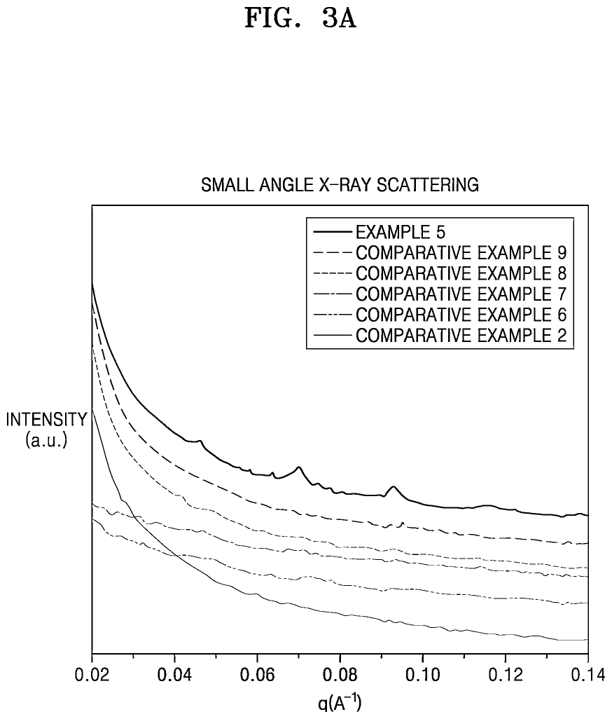

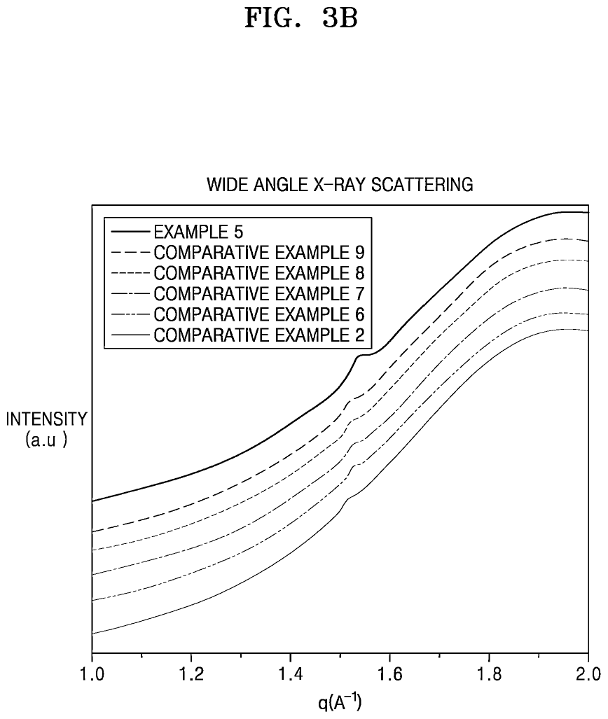

FIG. 3A is a small angle X-ray scattering graph of a cosmetic composition according to an embodiment.

As a result, as shown in FIG. 3A, no peak in small angle X-ray scattering was observed for Comparative Example 2, which does not contain a fatty acid, or for Comparative Example 6 to Comparative Example 9, which include one type of fatty acid. On the other hand, for Example 5 including both unsaturated fatty acid and saturated fatty acid, q values were observed at 0.046 Å⁻¹, 0.070 Å⁻¹, 0.093 Å⁻¹, 0.115 Å⁻¹, and 0.138 Å⁻¹ with a ratio of 2:3:4:5:6 with 0.046 Å⁻¹ as the second peak. The interlayer distance was confirmed to be about 27 nm when measured by substituting Bragg's equation.

In other words, it may be seen that the cosmetic composition according to an aspect has a lamellar structure similar to the stratum corneum structure of the skin.

FIG. 3B is a wide angle X-ray scattering graph of a cosmetic composition according to an embodiment.

As a result, as shown in FIG. 3B, a peak at 1.52 Å⁻¹ was observed for Example 5 and Comparative Example 6 to Comparative Example 9, confirming that they are flexible hexagonal packing structures with a d-spacing of 0.414 nm according to Bragg's equation.

In other words, it may be seen that the cosmetic composition according to an aspect, by containing the ceramide and fatty acid, form a lamellar structure, and especially when the cosmetic composition include both saturated fatty acid and unsaturated fatty acid, the cosmetic composition is conducive to the formation of a lamellar structure because fatty acids with a different alkyl group are closely arranged at the emulsion interface.

### Experimental Example 3. Skin Permeation Evaluation

A Franz diffusion cell system was utilized to evaluate the skin absorption effect of an active ingredient of a cosmetic composition according to an aspect. Specifically, Example 6, which each include a certain amount of niacinamide, a whitening functional ingredient, and adenosine, a wrinkle functional ingredient, and Comparative Example 10, which include niacinamide, were applied on an artificial membrane (Strat-MR membrane, Merck) or a human skin model (EpiTEM, ROKIT Healthcare). A receptor chamber was filled with 7 mL of PBS (pH 7.4) as a receptor phase to allow niacinamide to permeate, and the experimental temperature was maintained at 32° C. using a water jacket and stirring bar. 2 hours, 4 hours, 8 hours, and 24 hours after the formulation was applied, the receptor phase was collected using a sampling port, and the amount of niacinamide that permeated the artificial membrane or human skin model was analyzed by high performance liquid chromatography (HPLC) from the collected sample. The HPLC analysis condition was shown in Table 5 below.

TABLE 5

| | |
|---|---|
| Column | C15 (250 × 4.6 mm, 5 μm, 300 A Jupiter) |
| Detector | Reversed-phase high-pressure liquid |
| | chromatography (UltiMate 3000, Dionex) |
| Flow rate | 1.0 mL/min |
| Absorbance | 263 nm |
| Mobile phase | Acetonitrile:Monopotassium phosphate = 3:97 |

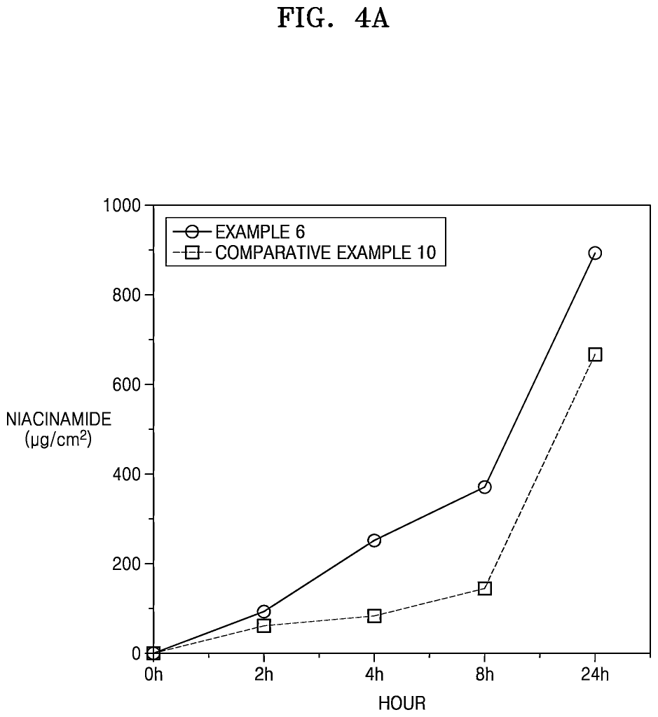

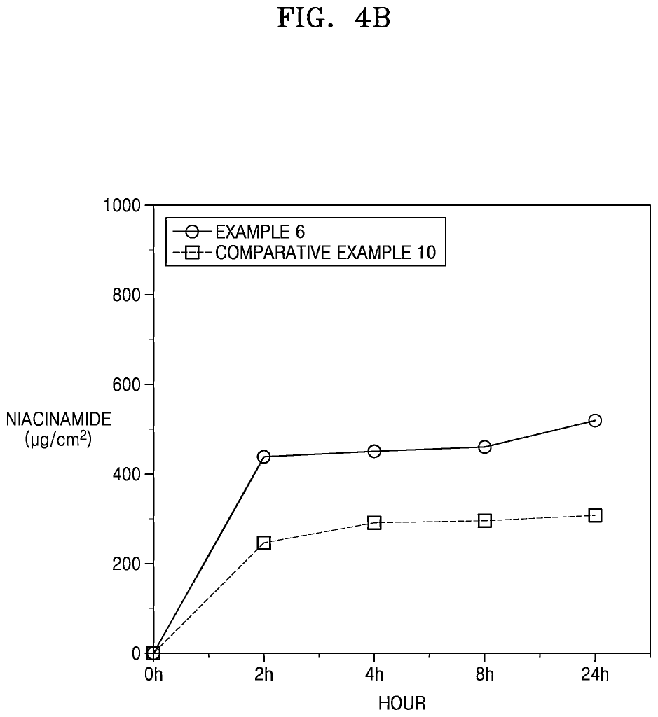

FIG. 4A and FIG. 4B are graphs confirming the effect of improving skin permeation through an artificial membrane (strat-M) and a human skin model (Epi-TEM), respectively, of a cosmetic composition according to an embodiment.

As a result, as shown in FIG. 4A, it may be determined that in the case of Example 6, the permeation amount of niacinamide in the skin after 24 hours is 892 μg/cm², indicating a cumulative permeation amount that is 1.34 times or more higher than that of Comparative Example 10, which is 664 μg/cm². In addition, as shown in FIG. 4B, in the case of Example 3, it may be determined that the permeation amount of niacinamide into the skin after 24 hours of niacinamide is 5206 μg/cm², which is more than 1.68 times higher than the cumulative permeation amount of Comparative Example 10, which is 3096 μg/cm².

In other words, the cosmetic composition according to an aspect may promote skin absorption of the active ingredient by including ceramide and fatty acid.

### Experimental Example 4. Skin Stability Evaluation

In order to confirm the skin stability of the cosmetic composition according to an asect, 33 adult males and females (mean age 35.5 years+1.82 years, 10 males, 23 females) without skin diseases were tested to evaluate the degree of irritation of the formulation prepared in Example 6 and Comparative Example 10 as follows. Specifically, Example 6 and Comparative Example 10 were applied to a patch in the center of an IQ Ultra Chamber™ (Chemotechnique Diagnostics, Sweden) in an amount of 20 μl each, and the patch was secured to the back with non-irritating tape for 24 hours. Skin reactions at 30 minutes and 24 hours after removal of the patch were performed according to the International Contact Dermatitis Research Group (ICDRG) reading criteria and the measurement evaluation method devised by Frosch & Kligman. The average skin reactivity of the test subjects obtained according to the judgment criteria was evaluated as hypoallergenic if the value was less than 0.9, mild irritation if the value was less than 2.9, moderate irritation if the value was less than 4.9, and strong irritation if the value was 5.0 or more, and the results were shown in Table 6 below.

TABLE 6

| | | | | Average | |
|---|---|---|---|---|---|
| | Number of | After 30 | After 24 | skin | |
| Column | responders | minutes | hours | reactivity | Verdict |
| Example 6 | 0 | 0 | 0 | 0 | Hypoallergenic |
| Comparative | 0 | 0 | 0 | 0 | Hypoallergenic |
| Example 10 | | | | | |

As a result, as shown in Table 6, it was confirmed that almost no skin irritation occurred in Example 6 and Comparative Example 10. Therefore, the cosmetic composition according to an aspect may minimize skin irritation by including hydrogenated lecithin, and may be suitable for use on sensitive skin.

### Experimental Example 5. Confirmation of Wrinkle Improvement Effect

A wrinkle improvement effect of the cosmetic composition according to an aspect was confirmed. Specifically, 20 adult subjects aged 30 years to 60 years (52.5 years+5.12 years) with wrinkles in the eye area and a global photodamage score of 3 or more were randomly assigned to use Example 6 and Comparative Example 10 twice daily for 4 weeks on both faces. Wrinkles in the periorbital region of both faces were measured using ANTERA 3D® CS (Miravex, Ireland) on the same areas before use (week 0), after 2 weeks of use, and after 4 weeks of use to assess the degree of reduction in overall size, depth, and maximum depth of wrinkles.

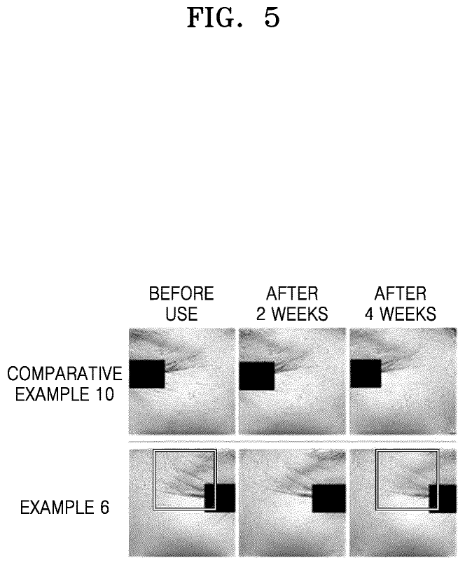

FIG. 5 is a photograph confirming the wrinkle improvement effect of the cosmetic composition according to an embodiment.

As a result, as shown in FIG. 5, in the case of Comparative Example 10, it was confirmed that there was almost no change in wrinkles around the eyes before use and after 2 weeks and 4 weeks. On the other hand, in the case of Example 6, it was confirmed that wrinkles around the eyes were reduced after 2 weeks of use compared to before use, and wrinkles around the eyes was confirmed to be significantly reduced after 4 weeks.

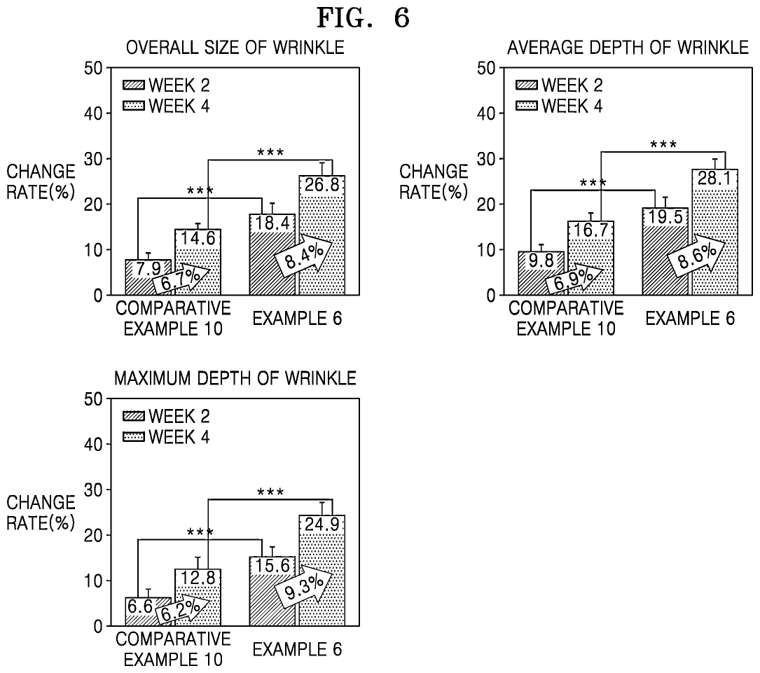

FIG. 6 is a graph confirming the change rate of the overall size of wrinkles, the average depth of wrinkles, and the maximum depth of wrinkles before and after using the cosmetic composition according to an embodiment.

As a result, as shown in FIG. 6, in the case of Example 6, it was confirmed that the overall reduction rate of the wrinkle factor increased compared to Comparative Example 10. Specifically, in the case of Example 3 compared to Comparative Example 10, it was confirmed that the overall size, depth, and maximum depth of wrinkles significantly decreased according to the increase of usage time, and each decreased by up to 26.8%, 28.1%, and 24.9% before and after use.

Thus, by containing lecithin and fatty acid, the cosmetic composition according to the present disclosure may restore elasticity and revitalize dry or damaged skin. In particular, when both saturated fatty acid and unsaturated fatty acid are included, the cosmetic composition may be stably absorbed deep into the skin due to the unsaturated fatty acid similar to a skin lipid composition, and thus may have an excellent moisturizing effect.

### Experimental Example 6. Confirmation of Skin Efficacy Enhancement Effect (1)

An effect of enhancing skin efficacy by promoting skin absorption of the cosmetic composition according to an aspect was confirmed. Specifically, 40 μg of each of Example 6 and Comparative Example 10 were applied on a Reconstructed Human Pigmented Epidermis-Brown (Phototype VI). After 24 hours, sections were prepared using Fontana-Masson staining, and a whitening improvement effect was evaluated by inhibiting the migration of melanin present in the basal layer of the human skin model to the epidermal layer through microscopic observation.

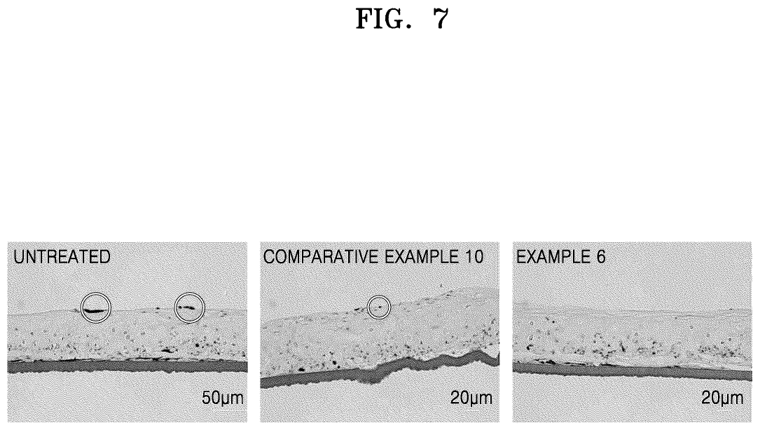

FIG. 7 is a photograph confirming the effect of enhancing skin efficacy of a cosmetic composition according to an embodiment.

As a result, as shown in FIG. 7, in the case of an untreated control group, it was confirmed that melanin formed from melanocytes migrated to the epidermal layer. On the other hand, in the case of Example 6 and Comparative Example 10 including niacinamide, it was confirmed that the migration of melanin was inhibited. In particular, in the case of Example 3 containing ceramide and fatty acid, it was confirmed that the melanin migration inhibition effect was superior to Comparative Example 10, which did not contain fatty acid.

That is, the cosmetic composition according to an aspect may effectively deliver skin absorption of the active ingredient by containing the fatty acid, thereby enhancing skin efficacy.

### Experimental Example 7. Confirmation of Skin Efficacy Enhancement Effect (2)

The whitening improvement effect of the cosmetic composition according to an aspect was confirmed. Specifically, 22 adult subjects aged 30 years to 60 years (44.2 years±6.42 years) with hyperpigmented areas were randomly assigned to use Example 6 and Comparative Example 10 twice daily for 4 weeks on both faces. Hyperpigmented areas on both sides of the face were measured using a Mexameter® MX18 (MDD4, Courage+Khazaka electronic GmbH, Germany) on the same areas before (week 0), after 2 weeks of use, and after 4 weeks of use to assess the degree of reduction in skin melanin index. In addition, RBX® images were extracted from photographs taken with VISIA CR (Canfield, USA) before use (0 weeks), 2 weeks after use, and 4 weeks after use, and analyzed with image J (National institutes of Health, USA), and the area recognized as hyperpigmented was obtained to evaluate the degree of reduction.

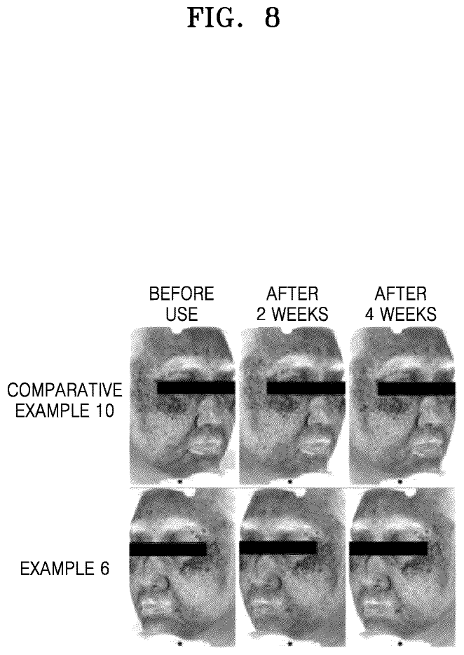

FIG. 8 is a photograph confirming the effect of reducing melanin pigment of a cosmetic composition according to an embodiment.

As a result, as shown in FIG. 8, in the case of Example 6, it was confirmed that the melanin pigment content of the entire face was significantly reduced before and after 2 and 4 weeks of use compared to Comparative Example 10.

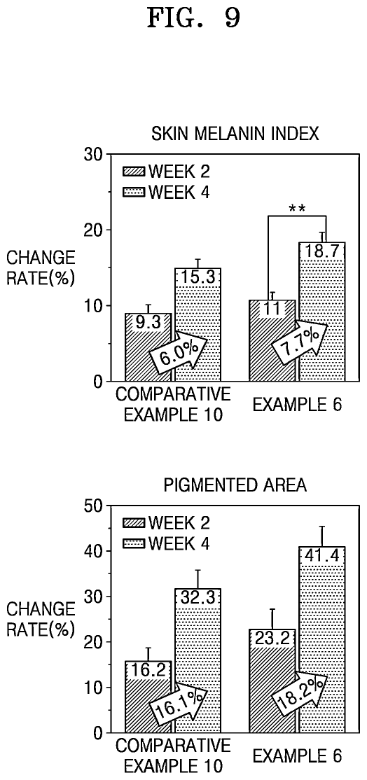

FIG. 9 is a graph confirming the change rate of the melanin index and pigmentation area (pixel) before and after using a cosmetic composition according to an embodiment.

As a result, as shown in FIG. 9, in the case of Example 6, it was confirmed that the overall reduction rate of the melanin index increased compared to Comparative Example 10. Specifically, in the case of Example 6 compared to Comparative Example 10, the melanin index and pigmented area (pixel) decreased significantly according to the increase of usage time, and it was confirmed that the melanin index and pigmented area (pixel) decreased by up to 7.7% and 18.2%, respectively, compared to before use.

In other words, the cosmetic composition according to an aspect may have the effect of enhancing skin effectiveness by including the fatty acid similar to the lipid composition of the skin, thereby helping the active ingredient to be stably absorbed deep into the skin.

The foregoing description of the disclosure is for illustrative purposes only, and one that has ordinary skill in the art to which the disclosure belongs will understand that it may be readily adapted to other specific forms without altering the technical ideas or essential features of the disclosure. Therefore, the embodiments described above should be understood in all respects as illustrative and not restrictive.

1. A cosmetic composition comprising a nanoparticle carrier comprising ceramides; hydrogenated lecithin; a fatty acid containing stearic acid and oleic acid; and a cholesterol derivative.

2. The cosmetic composition of claim 1, wherein the ceramides are comprised in an amount of 0.01 wt % to 0.1 wt %, based on the total weight of the composition.

3. The cosmetic composition of claim 1, wherein the hydrogenated lecithin is comprised in an amount of 0.1 wt % to 0.4 wt %, based on the total weight of the composition.

4. The cosmetic composition of claim 1, wherein the oleic acid is comprised in an amount of 0.01 wt % to 0.07 wt %, based on the total weight of the composition.

5. The cosmetic composition of claim 1, wherein the stearic acid and oleic acid are comprised in an amount of 0.01 wt % to 0.2 wt %, based on the total weight of the composition.

6. The cosmetic composition of claim 1, wherein the stearic acid and oleic acid are mixed in a weight ratio of 1:0.01 to 99.

7. The cosmetic composition of claim 1, wherein the cholesterol derivative is comprised in an amount of 0.05 wt % to 0.2 wt %, based on the total weight of the composition.

8. The cosmetic composition of claim 1, wherein the nanoparticle carrier is one that has a size of 100 nm to 300 nm.

9. The cosmetic composition of claim 1, further comprising a skin active substance selected from the group consisting of niacinamide, adenosine, Centella asiatica, oleanolic acid, retinol, ascorbic acid, flavonoids, polyphenols, and isoflavones.
